(19) United States
(12) Patent Application Publication
(10) Pub. No.: US 2024/0260945 A1
(43) Pub. Date: Aug. 8, 2024

(54) FOUR-DIMENSIONAL LUNG ULTRASOUND IMAGING FOR IMAGE-GUIDED INTERVENTIONAL PROCEDURES

(71) Applicant: Broncus Medical Inc., San Jose, CA (US)

(72) Inventors: Maysam Shahedi, Plano, TX (US); Kun-Chang Yu, San Jose, CA (US); Thomas M. Keast, Sunnyvale, CA (US)

(21) Appl. No.: 18/416,063

(22) Filed: Jan. 18, 2024

Related U.S. Application Data
(60) Provisional application No. 63/443,266, filed on Feb. 3, 2023.

(51) Int. Cl. A61B 8/08; A61B 8/00; A61B 34/20; G06T 7/33; G16H 30/20

(52) U.S. Cl. CPC A61B 8/5284; A61B 8/0841; A61B 8/4263; A61B 8/4488; A61B 8/466; A61B 8/5261; A61B 8/58; A61B 34/20; G06T 7/337; G16H 30/20; A61B 2034/2055; A61B 2034/2063; G06T 2207/10081; G06T 2207/10088; G06T 2207/10132; G06T 2207/30061

(57) ABSTRACT

Described herein are systems for 4D imaging of the lung based on live 2D ultrasound images. An ultrasound probe generates 2D image data of the lung during a plurality of breathing cycles. A computer is programmed and operable to group the 2D image data into subsets based on their point (or optionally phase) in the breathing cycle, and to reconstruct the 2D image data subsets into a 3D image volume based on the location information of the probe. Optionally, the 3D reconstructed image volume is displayed with target and route information. In embodiments, the computer is further operable to detect and track a surgical device in the lung such as an aspiration needle and to display the surgical device in the 3D reconstructed image volume for assisting the physician reach the target. Related methods are also described.

### CROSS-REFERENCE TO RELATED APPLICATIONS

This claims priority to provisional patent application No. 63/443,266, filed Feb. 3, 2023, and entitled “FOUR-DIMENSIONAL LUNG ULTRASOUND IMAGING FOR IMAGE-GUIDED INTERVENTIONAL PROCEDURES”.

### BACKGROUND OF THE INVENTION

Fluoroscopy imaging is commonly used in various diagnostic and therapeutic procedures such as transthoracic needle biopsy (TTNA), transbronchial needle aspiration (TBNA), and lung tumor ablation. Fluoroscopy imaging assists in targeting the correct tissue or navigating the bronchoscope catheter to the correct airway. Notwithstanding this benefit, fluoroscopy exposes the patients to ionizing radiation. This is undesirable.

Simply prohibiting the use of the fluoroscopy is not a good option because of the value added to the procedure by using fluoroscopy. Fluoroscopy imaging provides accurate imaging of the target as the lung and target move during breathing. Without an accurate intra-operative image, an accurate estimation of the target location during the procedure is not available.

Another potential imaging modality is ultrasound. However, there are several challenges associated with ultrasound imaging of the lung, not the least of which is the lung motion, image shadowing artifacts and beam scattering due to major airways and alveoli, and the limited acoustic window between the ribs. Due to these challenges, ultrasound imaging of the lung is not an available solution.

A system and method as described herein is therefore still desirable.

### SUMMARY OF THE INVENTION

In embodiments of the invention, a system for four-dimensional (4D) imaging of the lung based on live two-dimensional (2D) ultrasound images includes an ultrasound probe for generating 2D image data of the lung during a plurality of breathing cycles. The system further includes a computer programmed and operable to group the 2D image data into subsets based on their point (or optionally phase) in the breathing cycle, and reconstruct the 2D image data subsets into a 3D image volume based on location information of the probe.

In embodiments of the invention, the probe and body motion are tracked, and preferably tracked by an optical-based tracking system.

In embodiments of the invention, the computer is further operable to register the 3D reconstructed image volume with a pre-operative 3D image volume of the patient's lung.

In embodiments, the registration is at least two-fold including an initial “coarse” image registration followed by a fine-tuning registration. The initial coarse registration is preferably non-iterative (and thus relatively fast) between a 3D image patch from the pre-operative image and the reconstructed ultrasound image. In embodiments, a set (preferably four or more) of biomarkers (e.g., pleura, blood vessels, calcifications, ribs, solid pulmonary nodules, etc.) that are visible on both image volumes are selected. A warping method (e.g., a thin plate spline warping method) is used to register the images based on matching the biomarker pairs. Examples of a thin plate spline warping method are described in Bookstein, Fred L. “Principal warps: Thin-plate splines and the decomposition of deformations.” IEEE Transactions on pattern analysis and machine intelligence 11, no. 6 (1989): 567-585.

Next, in preferred embodiments, a fine-tuning step is performed. The second registration step is a more exhaustive iterative image registration approach (with limited freedom in transforming or warping the moving image) to fine-tune the image registration and reduce the registration error. Examples of suitable iterative approaches for the fine-tuning step are described in Lange, Thomas, Nils Papenberg, Stefan Heldmann, Jan Modersitzki, Bernd Fischer, Hans Lamecker, and Peter M. Schlag. “3D ultrasound-CT registration of the liver using combined landmark-intensity information.” International journal of computer assisted radiology and surgery 4 (2009): 79-88; and Wein, Wolfgang, Barbara Roper, and Nassir Navab. “Automatic registration and fusion of ultrasound with CT for radiotherapy.” In Medical Image Computing and Computer-Assisted Intervention-MICCAI 2005: 8th International Conference, Palm Springs, CA, USA, Oct. 26-29, 2005, Proceedings, Part II 8, pp. 303-311. Springer Berlin Heidelberg, 2005.

In embodiments of the invention, the computer is further operable to compute a route, and the 3D reconstructed image volume is displayed with target and route information.

In embodiments of the invention, the computer is further operable to detect and track a surgical device and to display the surgical device in the 3D reconstructed image volume for assisting the physician reach the target. Optionally, the surgical device is an aspiration needle or ablation probe or catheter.

In embodiments of the invention, the processor is further programmed and operable to initially or periodically register the surgical device (e.g., aspiration needle or ablation probe) location with the pre-operative 3D image data coordinate system based on moving the surgical device to a known lung biomarker or landmark that is within the ultrasound probe's field of view and preferably, with no airway or bony structure blockage.

In embodiments of the invention, the processor is further programmed and operable to compute a suggested location, and optionally a suggested angle, for the ultrasound probe for generating the 2D image slices. In embodiments of the invention, the processor computes the suggested location and angle based on location of the targets (e.g., pulmonary nodules) and airways in the lung, lung motion, and position of the ribs or other bony structures.

In embodiments of the invention, the system is operable to display the suggested location and angle, and optionally alert the physician if the probe is off-track or off-angle. Examples of types of alerts include visual and audible.

In embodiments of the invention, a method generates 4D ultrasound imaging of the lung using a 2D ultrasound transducer. The method comprises tracking the position and orientation of the ultrasound transducer. The method further comprises tracking the patient chest for estimating the phases of breathing. The method further comprises storing, for each 2D ultrasound slice, position information and the breathing phase associated with each image that has been acquired. The method further comprises grouping the image slices that are related to the same phase of breathing by phase, and computing one 3D image volume per phase.

In embodiments of the invention, the method comprises grouping the image slices that are related to the same point in the breathing cycle by point, and computing one 3D image volume per point.

In embodiments of the invention, a 4D ultrasound imaging system comprises an ultrasound scanner; an ultrasound transducer (e.g., a linear or phased-array ultrasound transducer); a position tracker; two sets of tracking markers (one for the transducer and one for patient's body); an image integration software module to reconstruct 3D image volumes out of 2D image slices; and optionally, an inter-modality image registration software module for 3D pre-operative image to 3D intra-operative ultrasound image registration. Examples of 3D pre-operative image include without limitation CT and MRI 3D image data sets of the patient generated prior to the procedure.

In embodiments of the invention, a pre-operative 3D image is deformed and aligned to the 3D ultrasound image for the current phase of breathing. The target location is mapped from the pre-operative 3D image to the intra-operative 4D ultrasound reconstructed image. This provides the physician a better estimation of the target location at each breathing phase for inserting a surgical device such as a biopsy needle or delivering the therapy. Examples of therapy include, without limitation, ablation, drug delivery, and excision or removal of the suspect lesion or nodule.

In embodiments of the invention, the method further comprises detecting and tracking a surgical device being advanced in the lung, and computing the location of the surgical device in the 3D reconstructed volume, and displaying the surgical device in the 3D reconstructed volume.

In embodiments of the invention, the method further comprises computing a route to the target or region of interest, and displaying the route in the 3D reconstructed volume.

In embodiments of the invention, the method further comprises receiving pre-acquired 3D image data of the lung of the patient over at least one breathing cycle, and registering the pre-acquired 3D image data of the lung to the 3D reconstructed volume including registering lung structures not visible in ultrasound.

In embodiments of the invention, the method further comprises generating a deformation lung model to register the pre-acquired 3D image data of the lung to the 3D reconstructed volume including registering lung structures not visible in ultrasound. In some embodiments, the deformation lung model is based on pre-operative data from multiple patients, and optionally, the model is a machine learning model.

In embodiments of the invention, the system is programmed and operable to receive pre-acquired 3D image data of the lung of the patient over at least one breathing cycle, and register the pre-acquired 3D image data of the lung to the 3D reconstructed volume including registering lung structures not visible in ultrasound.

In embodiments of the invention, the system is programmed and operable to generate a deformation lung model to register the pre-acquired 3D image data of the lung to the 3D reconstructed volume including registering lung structures not visible in ultrasound. In some embodiments, the deformation lung model is based on pre-operative data from multiple patients, and optionally, the model is a machine learning model.

### Objects and Advantages of the Invention

An object of the invention is to collect 2D image slices of the lung of a patient using an ultrasound probe, and to track the location and orientation of the ultrasound probe as the 2D image slices are being generated.

Another object of the invention is to acquire the 2D image slices over a plurality of breathing phases of the patient.

Another object of the invention is to group the 2D image slices into sets by a specific point or phase of the breathing cycle, and reconstruct each set into a 3D image volume.

Another object of the invention is to use the 3D imaging methods and systems described herein intra-operatively and optionally for image-guided procedures.

Another object of the invention is to compute an estimate of the optimal location and angle of the ultrasound transducer for imaging and to mitigate the airways' effects on the ultrasound images based on anatomical information obtained from the pre-operative image.

The description, objects and advantages of the present invention will become apparent from the detailed description to follow, together with the accompanying drawings.

### BRIEF DESCRIPTION OF THE DRAWINGS

FIG. 1 is a flow diagram of a process for performing 3D ultrasound imaging of a lung in accordance with an embodiment of the invention.

FIG. 2 is a schematic illustration showing 3D imaging of the lung of a patient holding their breath using an optically tracked 2D ultrasound transducer (shown from the sagittal plane) in accordance with an embodiment of the invention.

FIG. 3 is a schematic illustration showing 3D image reconstruction using an optically tracked 2D ultrasound transducer (moving in the transverse plane) in accordance with an embodiment of the invention.

FIG. 4 is a block diagram of a system for generating 3D images of the lung based on 2D ultrasound in accordance with an embodiment of the invention.

FIG. 5 is a flow diagram of a process for performing 4D ultrasound imaging of a lung in accordance with an embodiment of the invention.

FIG. 6 is a schematic illustration showing 4D imaging of the lung of a patient while breathing using an optically tracked 2D ultrasound transducer (shown from the sagittal plane) in accordance with an embodiment of the invention.

FIG. 7 is another schematic diagram of 4D ultrasound imaging of a lung of a patient while breathing using an optically tracked 2D ultrasound transducer (shown from the transverse plane) in accordance with an embodiment of the invention.

FIG. 8 is a flow diagram of a process for registering the 4D ultrasound imaging with pre-operative CT image data in accordance with an embodiment of the invention.

FIG. 9 is a schematic diagram for registering the 4D ultrasound imaging with pre-operative CT image data in accordance with an embodiment of the invention.

FIG. 10 is a flow diagram of a process for assisting a physician guide an instrument to a target in the lung based on live 2D ultrasound imaging in accordance with an embodiment of the invention.

### DETAILED DESCRIPTION OF THE INVENTION

Before the present invention is described in detail, it is to be understood that this invention is not limited to particular variations set forth herein as various changes or modifications may be made to the invention described and equivalents may be substituted without departing from the spirit and scope of the invention. As will be apparent to those of skill in the art upon reading this disclosure, each of the individual embodiments described and illustrated herein has discrete components and features which may be readily separated from or combined with the features of any of the other several embodiments without departing from the scope or spirit of the present invention. In addition, many modifications may be made to adapt a particular situation, material, composition of matter, process, process act(s) or step(s) to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Last, it is to be appreciated that unless defined otherwise, all technical and scientific terms used herein have the same meaning as commonly understood by one of ordinary skill in the art to which this invention belongs.

Described herein are systems and methods for 4D lung imaging based on 2D ultrasound images. In embodiments of the invention, the 3D and 4D imaging is registered with pre-acquired CT image data and optionally, can be used to assist a physician reach a target in the lung during a procedure.

### 3D Imaging Method

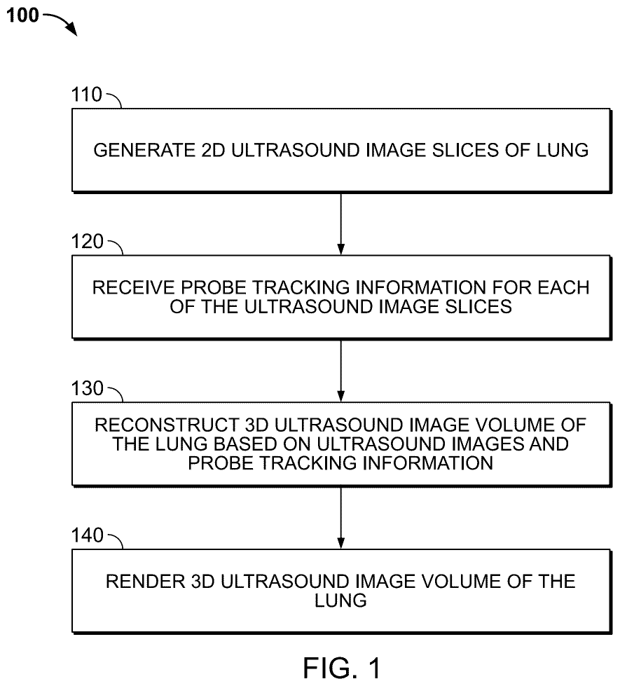

With reference to FIG. 1, an overview of an ultrasound imaging method 100 is shown for obtaining 3D imaging of the lung of a patient based on 2D ultrasound images in accordance with an embodiment of the invention.

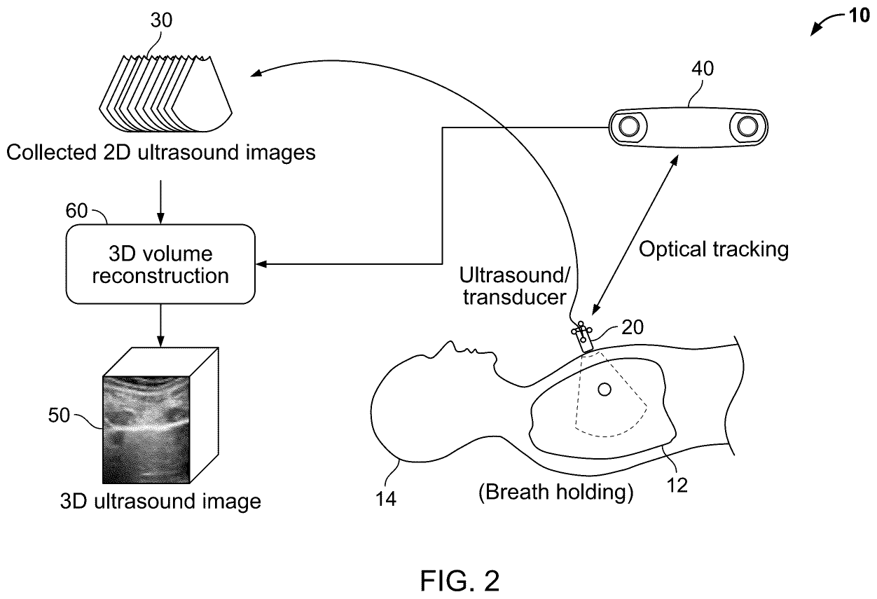

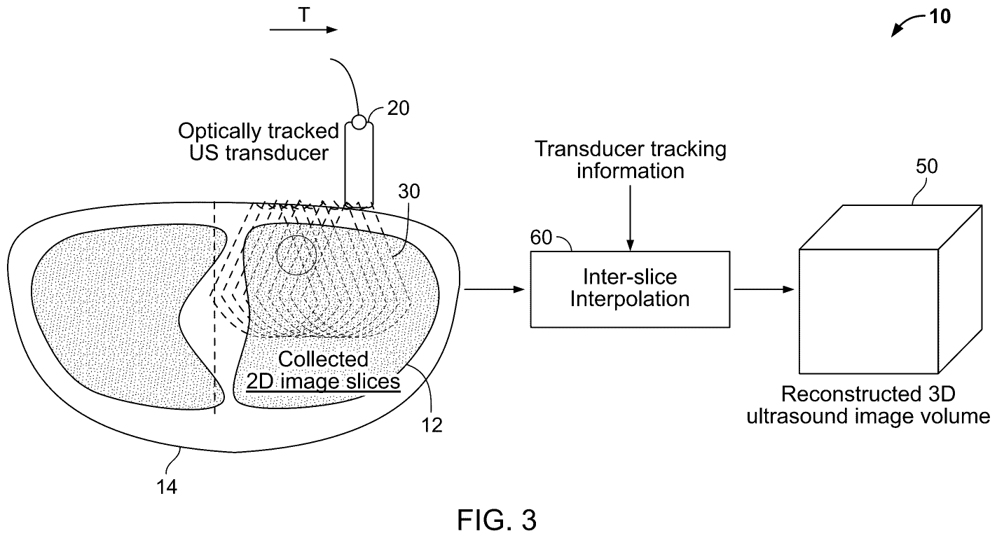

Step 110 states to generate 2D ultrasound image slices of the lung. With additional reference to FIGS. 2-3, this step may be performed by an ultrasound transducer or probe 20. The probe 20 is operable to obtain a set of 2D image slices 30 of the lung 12 by moving the probe across the patient 14 while the patient is holding their breath. In embodiments, the probe is moved transversely (T) by hand across the patient. An acoustically-conducting gel can be applied between the skin and probe to improve the signal. Examples of ultrasound transducers or probes include without limitation, a phased-array ultrasound transducer (e.g., a cardiac imaging transducer GE 3Sc-RS Phased Array Transducer, manufactured by GE Healthcare (Wauwatosa, WI).

Step 120 states to receive probe tracking information for each of the ultrasound image slices. With reference again to FIG. 2-3, the system preferably includes an optical tracking system 40 to track the location of the probe 20 as the probe is moved. Examples of probe tracking systems include without limitation, the Polaris Spectra or Vega tracker, both of which are manufactured by NDI in Waterloo, Ontario, Canada.

Step 130 states to reconstruct the 3D volume of the lung based on the set of 2D slices 30 and location input from the tracker 40. In embodiments, and with reference to FIG. 3, the 2D slices are assembled into 3D voxels using inter-slice interpolation. An exemplary technique for reconstructing the 2D slices into the 3D volume is described in, e.g., Chen, Zhenping, and Qinghua Huang. “Real-time freehand 3D ultrasound imaging.” Computer Methods in Biomechanics and Biomedical Engineering: Imaging & Visualization 6, no. 1 (2018): 74-83. However, it is to be understood that other techniques for reconstructing the ultrasound data into 3D data sets of the lung may be applied except where excluded by any appended claims.

Step 140 states to render the 3D ultrasound image volume of the lung. This step may be performed by a software renderer based on the 3D reconstructed volume from step 130. Optionally, and as described further herein, a wide range of views and overlays may be rendered and displayed based on user input. Exemplary views include 2D or 3D virtual views in which the physician can change the viewing angle, zoom, annotate, and store desired screen shots.

### 4D Ultrasound Imaging Method

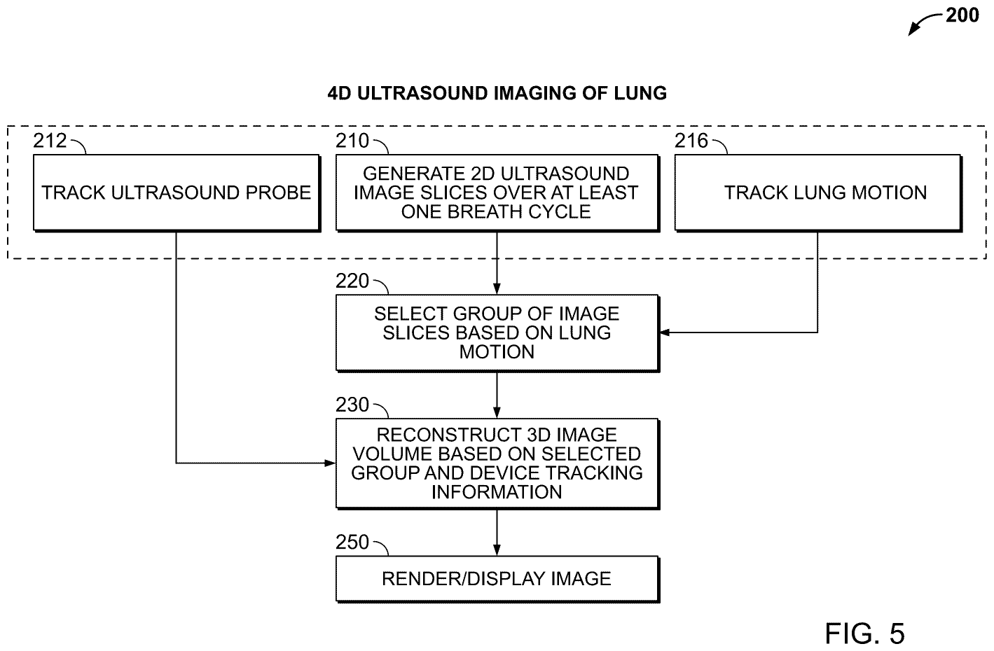

With reference to FIG. 5, an overview of an ultrasound imaging method 200 is shown for obtaining 4D imaging of the lung of a patient based on 2D ultrasound images in accordance with an embodiment of the invention.

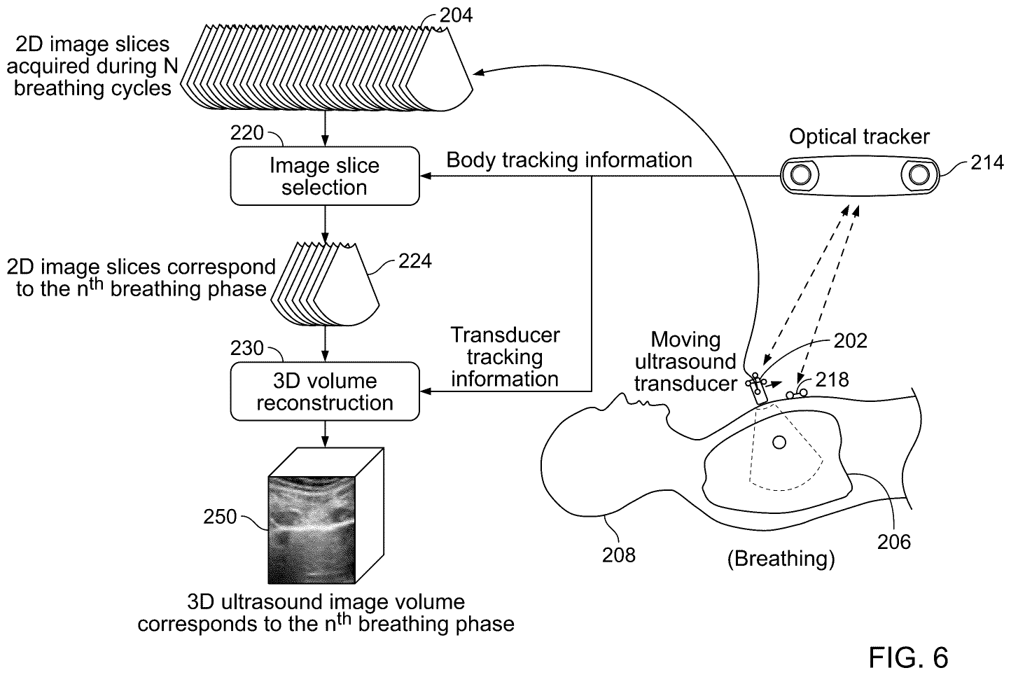

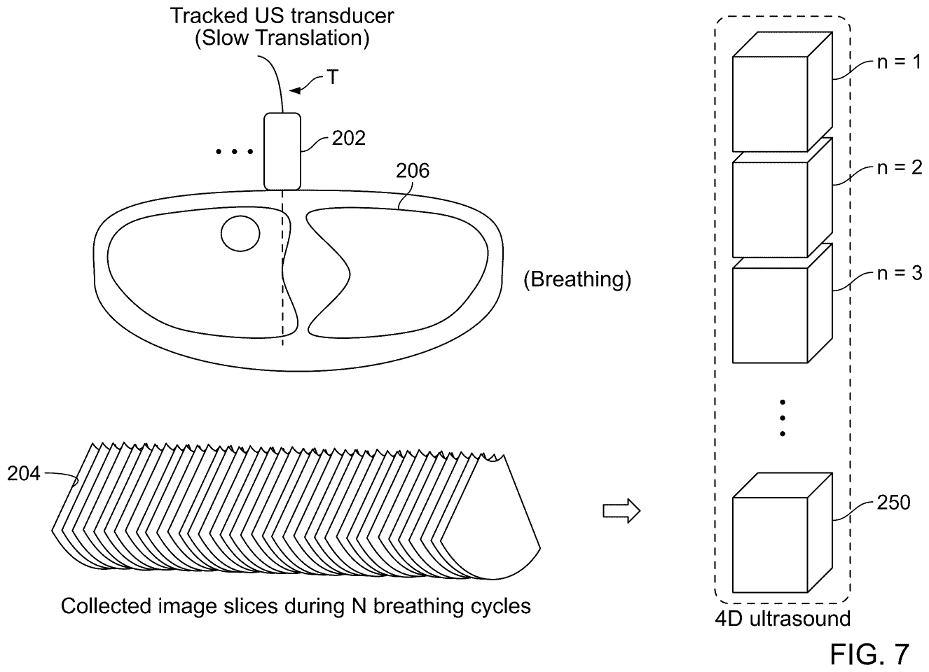

Step 210 states to generate 2D ultrasound image slices of the lung over multiple breath cycles. With additional reference to FIGS. 6-7, an ultrasound transducer or probe 202 is operable to obtain a set of 2D image slices 204 of the lung 206 by moving the probe across the patient 208 while the patient is breathing. In embodiments, the probe is moved transversely (T), longitudinally, and/or rotationally by hand across the patient. An acoustically-conducting gel can be applied between the skin and probe to improve the signal. The ultrasound transducer or probe 202 can be, but is not limited to, that described above in connection with FIGS. 1-3.

Step 212 states to track ultrasound probe. This step may be performed as described above in connection with FIGS. 1-3 in which the probe is tracked by an optical tracking system 214 to track the position of the probe as the probe is moved. The optical tracking system 214 can be, but is not limited to, that described above in connection with FIGS. 1-3.

Step 216 states to track lung motion. This step may be performed using an optical tracking system 214 as described above except the optical tracking system is used to track one or more markers 218 arranged on the patient's body. For example, as shown in FIG. 6, a marker 218 is placed on the patient's chest. As the chest moves due to the breathing cycle, the location of the marker and thus motion of the breathing cycle is recorded for each 2D image generated.

Step 220 states to select a group of image slices based on the lung motion. This step is performed by forming subsets of the 2D image slices from step 210 according to their point or phase in the breath cycle. Ultrasound 2D slices obtained at a common point in the breath cycle are grouped together as a subset 224 and can be defined as the subset 224 of image slices at the n^{th} breathing point of the patient's breathing cycle.

Step 230 states to reconstruct the 3D volume of the lung based on a selected group of 2D slices 224 from step 220 and probe tracker step 212. The grouped 2D slices 224 can be assembled into 3D voxels using inter-slice interpolation as described herein.

This process may be repeated for each subset of 2D image slices to reconstruct a sequence of 3D volumes over the patient's entire breathing cycle, namely, 4D imaging of the lung.

Step 250 states to render/display the image. This step may be performed by a software rendering module on a computer system based on the 3D reconstructed volume from step 230. Optionally, and as described further herein, a wide range of views and overlays may be rendered and displayed based on user input. Exemplary views include 2D or 3D virtual views in which the physician can change the viewing angle, zoom, annotate, and store a desired screen shot.

Additionally, in embodiments, the system is operable to accept user input to adjust the point in the breathing cycle at which to display or view the lung. For example, the physician may input to observe the lung at full inspiration, full expiration, or any point in-between full inspiration and full expiration. The system is operable to compute the image based on such user input given the previously reconstructed 3D model from step 230. Optionally, an image sequence can be computed and rendered according to chronological sequence. The lung video may be shown for a portion or phase of a breathing cycle, an entire breathing cycle, or across multiple breathing cycles. The system is operable to allow the physician to select a viewing angle, zoom, etc. and play/pause the sequence or video of the lung. The physician may fast-forward, reverse, speed up, slow down, pause, etc.

### Registration

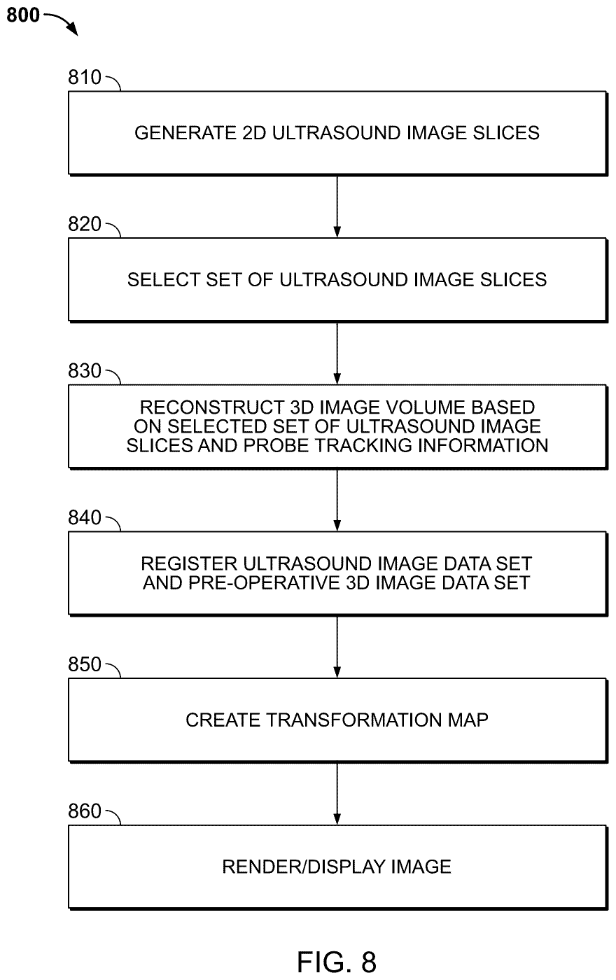

With reference to FIG. 8, an overview of an ultrasound imaging method 800 is shown for obtaining 4D imaging of the lung of a patient based on 2D ultrasound images, and registering the live imaging of the lung with pre-acquired image data of the lung in accordance with an embodiment of the invention.

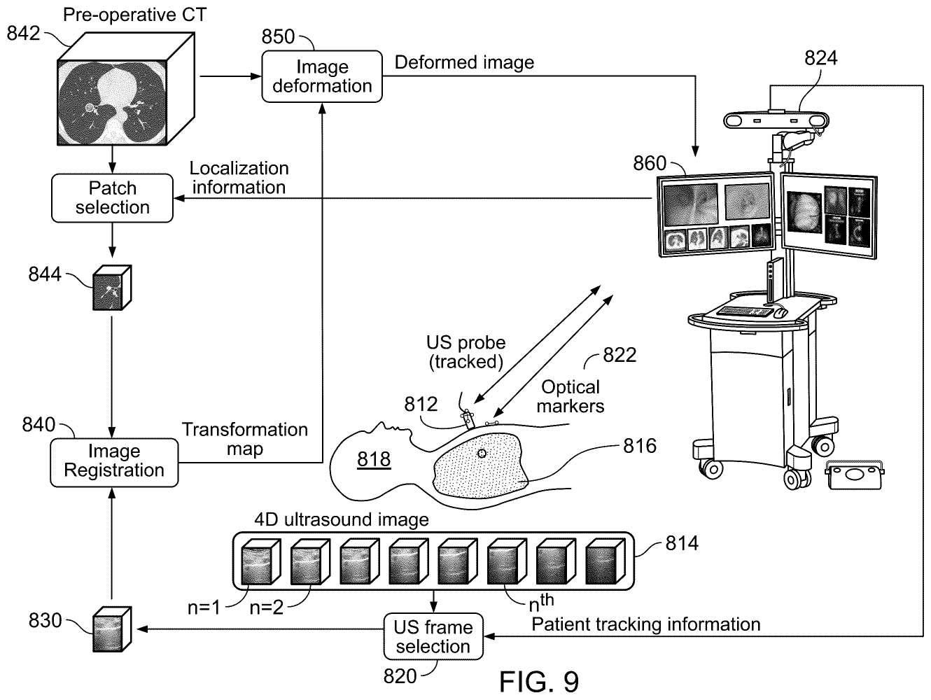

Step 810 states to generate 2D ultrasound image slices of the lung over at least one breath cycle, and preferably over multiple breath cycles. With additional reference to FIG. 9, an ultrasound transducer or probe 812 is operable to obtain a set of 2D image slices of the lung 816 by moving the probe across the patient 818 while the patient is breathing. In embodiments, the probe is moved transversely (T) or otherwise (e.g., rotationally) by hand across the patient. An acoustically-conducting gel can be applied between the skin and probe to improve the signal. The ultrasound transducer or probe 812 can be, but is not limited to, that described above in connection with FIGS. 1-3.

Tracking information is obtained for each 2D image slice including probe location information and body motion information. The tracking information can be obtained using an optical tracking system 824 and markers 822 placed on both the probe and the patient body, as described above in connection with FIG. 6. As the chest moves due to the breathing cycle, the location of the marker 822 and thus motion of the breathing cycle is recorded for each 2D image slice generated.

Step 820 states to select a group of image slices based on the lung motion. This step is performed by forming subsets of the 2D image slices from step 810 according to their point or phase in the breath cycle. 2D image slices are grouped together according to a common point in the breath cycle as a subset. The subset can be defined as a collection of 2D image slices at the n^{th} breathing point of the breathing cycle.

Step 830 states to reconstruct a 3D volume of the lung based on a selected group of 2D image slices from step 820. With reference again to FIG. 9, the subset of 2D slices 820 can be assembled into 3D image volume 830 (e.g., a set of voxels) using inter-slice interpolation as described herein. However, other techniques may be applied to reconstruct the 2D image slices into the 3D image volume as is known to those of skill in the art.

Step 840 states to register ultrasound image data set and the pre-operative 3D image data set. With reference again to FIG. 9, the 3D reconstructed image volume 830 is registered to the pre-operative 3D image data set 842. The pre-operative 3D image data 842 may include, for example, preoperative image data arising from CT, MRI, or other means.

Registration. Various types of registration techniques may be employed to register the 3D reconstructed image data (namely, the ultrasound generated data) with the pre-acquired 3D image data. In the embodiment shown in FIG. 9, for example, a patch-to-image matching algorithm is applied for registration. A patch 844 is selected from a corresponding region on the pre-acquired 3D image data set 842 for matching or registering with the reconstructed 3D image 830. Localization information 846 is provided to assist in defining the location of the image patch. Localization information may include, for example, breath phase estimate (e.g., full inspiration), view angle, presence of a landmark (e.g., bifurcation), marker, region of interest, target, number of control points, etc. After selecting the corresponding patch from the pre-acquired image, any one of server inter-modality 3D image registration techniques could be used such as, for example, the techniques described in: Rouet, Laurence, Cecile Dufour, Antoine Collet Billon, and K. Bredahl. “CT and 3D-ultrasound registration for spatial comparison of post-EVAR abdominal aortic aneurysm measurements: A cross-sectional study.” Computerized Medical Imaging and Graphics 73 (2019): 49-59; Masoumi, Nima, Yiming Xiao, and Hassan Rivaz. “ARENA: Inter-modality affine registration using evolutionary strategy.” International Journal of Computer Assisted Radiology and Surgery 14, no. 3 (2019): 441-450; lommi, David, Alejandra Valladares, Michael Figl, Marko Grahovac, Gabor Fichtinger, and Johann Hummel. “3D ultrasound guided navigation system with hybrid image fusion.” Scientific Reports 11, no. 1 (2021): 1-10; or Cool, Derek W., Xuli Zhang, Cesare Romagnoli, Jonathan I. Izawa, Walter M. Romano, and Aaron Fenster. “Evaluation of MRI-TRUS fusion versus cognitive registration accuracy for MRI-targeted, TRUS-guided prostate biopsy.” AJR Am J Roentgenol 204, no. 1 (2015): 83-91.

Calibration. In embodiments, the physician (or co-pilot) performs a manual synchronization or calibration-step to confirm registration of a candidate image of the pre-acquired 3D image data set 842 with a live 2D image slice or a reconstructed 3D image computed from the live 2D image slices. For example, the physician may move or adjust the ultrasound probe until the main carina (or another conspicuous landmark or bio marker) is visible in the live 2D ultrasound image. Examples of landmarks or markers include, without limitation, blood vessels, airways, and bronchi. Next, an image patch from the pre-operative image data set may be selected from the corresponding field of view of ultrasound imaging, and computed for the same landmark or biomarker. The physician may continue to move and adjust the location and angle of the probe 812 until the real time image matches the pre-operative image. Once the physician is satisfied there is a sufficient match, the physician confirms the position and location (and angle) information of the ultrasound probe. This calibration process can be repeated at additional known landmarks and biomarkers. The confirmed parameters are input to the registration algorithm or model described above, serving to make the ultrasound to pre-operative image registration more accurate and faster.

Next, and with reference to step 850, a transformation map is created corresponding to deforming the pre-operative 3D image data to match the 3D reconstructed image data 830. This process can be repeated to match additional 3D reconstructed image data arising from other selected subsets of the 2D ultrasound groups 814 corresponding to other points in the breath cycle. Indeed, the process can be repeated for a portion of the breath cycle, the entire breath cycle, or multiple breath cycles.

Step 860 states to render/display the image. This step may be performed by a software rendering module on a computer system based on the 3D reconstructed volume from step 830 and/or the deformed image from step 850. Optionally, and as described herein, a wide range of views and overlays may be rendered and displayed based on user input. Exemplary views include 2D or 3D virtual views in which the physician can change the viewing angle, zoom, annotate, and store screen shots.

Additionally, in the 4D imaging embodiment shown in FIGS. 8-9, the user can adjust the phase or point in the breathing cycle at which to view the lung. For example, the physician may input to observe the lung at full inspiration or full expiration, or any point in-between full inspiration and full expiration. The system is programmed and operable to compute the image based on such user input given the previously reconstructed 3D model from step 830 and/or the deformed image from step 850.

Optionally, an image sequence can be computed and rendered according to natural breathing or anticipated breathing motion. The lung may be shown during a portion of a breathing cycle, for the entire breathing cycle, or across multiple breathing cycles. The system is configured to allow the physician to select a viewing angle, zoom, and play the sequence or video of the lung. The system is configured to allow the physician to fast-forward, reverse, speed up, slow down, and pause the video.

### Guidance Method

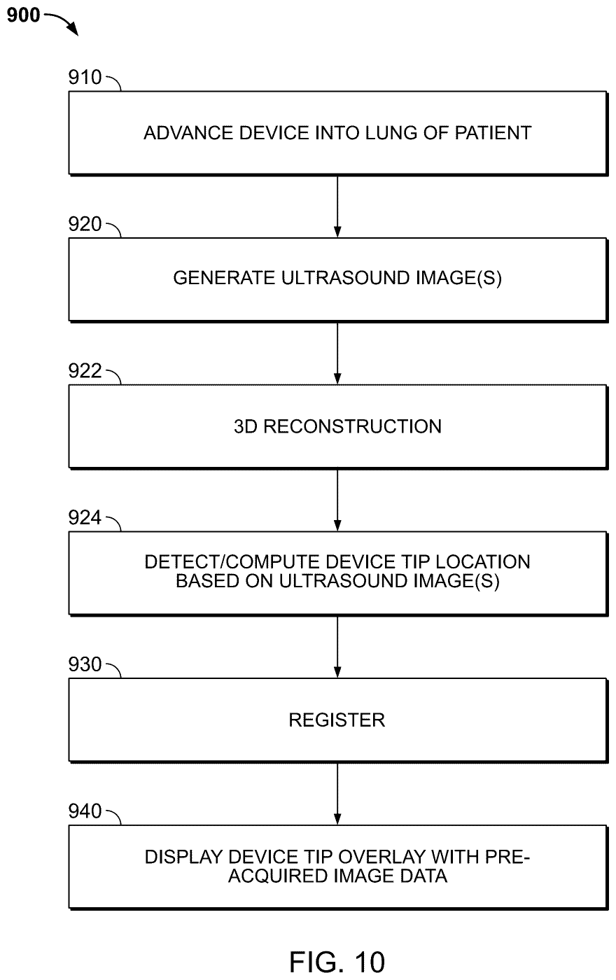

With reference to FIG. 10, an overview of an ultrasound guidance method 900 is shown for assisting a physician reach a target in the lung in accordance with an embodiment of the present invention.

Step 910 states to advance a device into the lung of a patient. In embodiments, a surgical device is passed through the subject's mouth, the trachea, a bronchi or more remote airways, and into the lung. The surgical device may vary. Examples of devices include, without limitation, catheters, sheaths, needles, ablation devices, stents, valves, fiducial markers, seeds, coils, etc. In embodiments, a surgical device or tool is advanced into the airways, and then through a wall of the airway towards a region of interest.

Additionally, in embodiments, the device is advanced percutaneously or transthoracically into the lung. For example, the invention includes assisting a physician perform a transthoracic needle aspiration or ablation (TTNA) in which the device is tracked in real time using the methods described herein.

Step 920 states to generate 2D ultrasound image slices of the lung. With reference again to FIG. 9, an ultrasound transducer or probe 812 is operable to obtain a set of 2D image slices of the lung 816 by moving the probe across the patient 818 while the patient is breathing. In embodiments, the probe is moved transversely (T) or otherwise (e.g., by rotation) by hand across the patient. An acoustically-conducting gel can be applied between the skin and probe to improve the signal. The ultrasound transducer or probe 812 can be, but is not limited to, that described above in connection with FIGS. 1-3.

Tracking information is obtained for each 2D slice including probe position information and body motion information. The tracking information can be obtained using an optical tracking system 824 and markers 822 placed on both the probe and the patient body, as described above. As the chest moves due to the breathing cycle, the location of the marker and motion of the breathing cycle is recorded. The position and motion can be recorded for each 2D image slice generated.

Step 922 states to reconstruct 3D volume. This step can be performed similar to step 830 shown in FIG. 8, and described above.

Step 924 states to compute device tip location. This step may be performed by detecting and tracking the device in the 3D reconstructed image. Machine learning detecting and tracking algorithms may be used to detect and track the surgical device. The surgical device may also include ultrasound visible markers and patterns to facilitate detection and location. Examples of suitable medical device detection and tracking techniques are described in Yang, Hongxu, Caifeng Shan, Alexander F. Kolen, and Peter H N de With. “Medical instrument detection in ultrasound-guided interventions: A review.” arXiv preprint arXiv:2007.04807 (2020).

Step 930 state to register the 3D reconstructed image volume with the pre-operative image data set. This step can be performed similar to step 840 shown in FIG. 8, and described above. The output of registration is a transformation map to deform the pre-operative 3D image data to match the instant 3D reconstructed volume including location coordinates for the surgical device detected in step 924.

Step 940 states to display the device tip in the 3D or 4D reconstructed image. This step may be performed by a software rendering module on a computer system based on the registered 3D ultrasound image data described above. For example, the system is programmed and operable to compute in the 3D reconstructed image, and for a user-input viewing angle, (a) the lung anatomies including, without limitation, airways, vessels, and optionally nerves (b) the region of interest or tumor location, and (c) the device tip location. As the physician advances the device tip, the device tip location is recomputed as described above and re-rendered—displaying the updated tip location information. In this manner, the method and system described herein can assist or guide the physician to reach a target. Optionally, a wide range of views and overlays may be rendered and displayed based on user input. Exemplary views include 2D or 3D virtual views in which the computer is operable to allow the physician to change the viewing angle, zoom, freeze frame, annotate, and save.

### Route Planning

Additionally, in embodiments, an optimal route to the target can be computed based on the pre-acquired 3D image data by the system, and overlaid onto the 3D reconstructed image that is displayed to the user. The route can be computed as described in, for example, U.S. Pat. No. 9,675,420, entitled “Methods and Apparatus for 3D Route Planning Through Hollow Organs”, to Higgins et al., and shown in LungPoint™ Lung Planning System and Archimedes™ Navigation System, both manufactured by Broncus Medical Inc. (San Jose, California). The computed route, and its location information, is stored with reference to the pre-acquired image data and coordinate system. When the pre-acquired data is registered with the live 3D ultrasound reconstructed image data, the route is likewise transposed to the 3D ultrasound reconstructed image data for display with the ultrasound 3D reconstructed volume.

In embodiments, the route is displayed on the CT-based rendered virtual bronchoscopic view wherein the CT image (and its segmentation labels such as, e.g., the airways and vessels) are warped based on the transfer function obtained through CT to 4D US registration. Then, the transform function is applied to the rendered bronchoscopic view at a desired point (e.g., in breathing cycle) and preferably in real time.

As the device tip location is computed and displayed as described above, the route can simultaneously be displayed or superimposed onto the lung image with the updated device location. In this embodiment, the physician can manipulate the surgical tool to follow the displayed route, and ultimately, navigate to the target.

### Block Diagram System

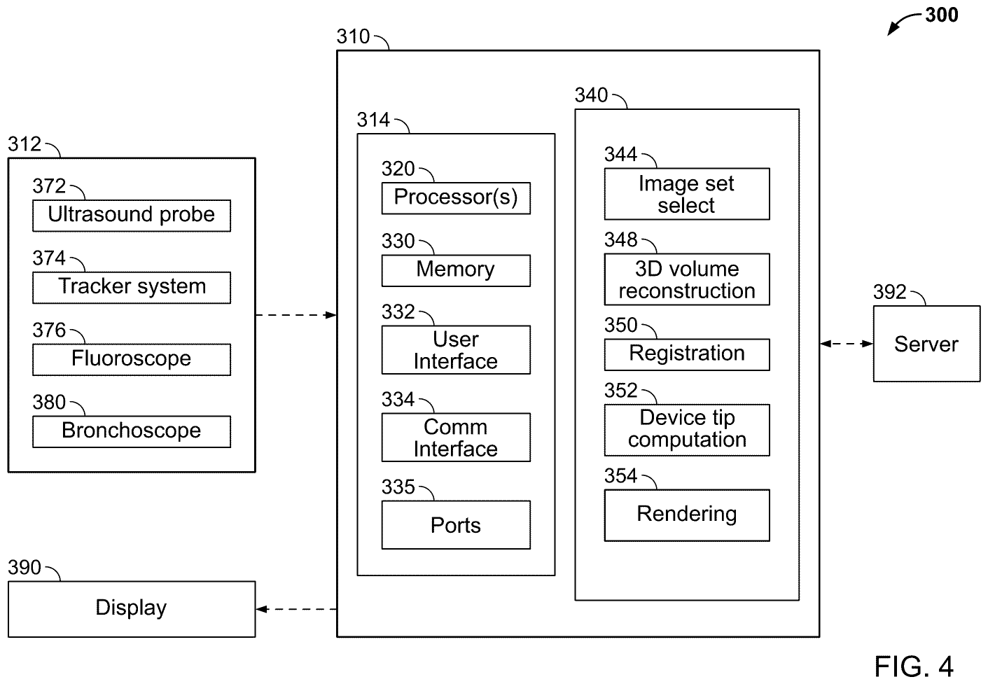

With reference to FIG. 4, a block diagram of a 4D ultrasound imaging and guidance system 300 is shown in accordance with an embodiment of the present invention.

The system 300 is shown including a workstation or computing device 310, various accessory devices 312 some of which are optional, a display 390, and an optional server 392 such as, e.g., a cloud-based server.

### Accessory Devices

The accessory devices 312 shown in FIG. 4 include ultrasound probes 372, tracker system 374, fluoroscope 376, and bronchoscope 380. However, it is to be understood that a wide variety of accessory devices may be incorporated or used with the system. As described herein the system includes ports and connectors for accessory devices and receiving data and input.

Examples of ultrasound probes 372 include 2D ultrasound probes or transducers, and preferably a phased array ultrasound transducer. The ultrasound probe generates live 2D image slices. The probe can be handheld and moved across the target area as described herein.

Examples of a tracker system 374 are the Polaris Spectra or Vega tracker, both of which are manufactured by NDI in Waterloo, Ontario, Canada. However, tracking information could also comprise location information arising from other types of technologies including, e.g., electromagnetic or GPS-types of device tracking systems such as Aurora, manufactured by Northern Digital (NDI) Inc. (Waterloo, ON, Canada). Regardless of the type of tracking system, it serves to generate position data of the ultrasound probe and the patient body motion. This location information is input to the workstation to compute the 3D reconstructed image, and optionally to provide physician guidance as described herein.

Examples of fluoroscopes 376 include C-Arm fluoroscopy machines such as the GE OEC 9800 C-Arm System manufactured by General Electric Company (United States). The live fluoro images or video can be sent to the workstation for confirming position and anatomy. In embodiments, the system is operable to register the live fluoro images with the 3D reconstructed image data and or pre-acquired image data set. Live fluoro can be useful for a physician to confirm location of the surgical device.

Examples of bronchoscopes 380 include flexible bronchoscopes such as the Olympus BF-XP160F EVIS EXERA Video Bronchoscope, manufactured by Olympus America. The live bronchoscopic images or video can be sent to the workstation. In embodiments, the system is operable to register the live bronchoscopic images with the 3D reconstructed image data and or pre-acquired image data set. Live bronchoscopic can be useful for a physician to assist in guidance and to confirm location of the surgical device.

FIG. 4 also shows computing device 310. The computing device 310 is shown having hardware 314 and software 340. In embodiments, the hardware includes at least one processor 320 (e.g., without limitation, a CPU, GPU, or another processing framework) operable to perform the software and processing operations described herein. An example of a suitable workstation is a Dell Computer Precision T5500 T5400, with Dual-core Intel Xeon 2.67 GHz processor, and a Nvidia Geforce GTX TITAN BLACK.

The computing device 310 is also shown having a storage or memory device 330 which can hold or store information including imaging, device, marker, and procedural data as well as one or more of the software modules 340, described herein. The memory device may be a non-transitory solid state storage device or hard drive. It is also to be understood, however, that although the system in FIG. 4 is shown with a memory 330 incorporated within the computing device 310 for receiving and storing various information, the invention is not so limited. In an alternative embodiment the system is configured to access a memory device such as a USB stick, a CD, or other portable media storage device.

The computing device 310 may also contain transitory or volatile storage including, e.g., Flash and RAM memory.

The computing device 310 is also shown including a user interface 332. Examples of user interface devices include, without limitation, a keyboard, joystick, and mouse. Examples of user input include a wide range of information such as device, anatomy, procedural, and patient data. The information can also include annotations or adjustments to data and objects as well as planning information and records. In a particular embodiment, the physician adjusts the phase of the breath cycle at which to view the 3D reconstructed ultrasound image.

The computing device 310 is also shown having a Comm (namely, communication) interface 334 or card which can be operable to communicate with various technologies including, e.g., Wi-Fi, Bluetooth, and UWB. The Comm interface(s) 334 may include a cellular communication interface.

The computing device 310 is also shown including one or more ports 335. Preferably the computing device is adapted to receive real-time images (e.g., ultrasound 372, fluoroscopy 376, and/or endoscopy 380 images) through various input ports or connectors 335 (e.g., USB port, video port, etc.). In embodiments, a frame grabber card captures individual video frames or images for processing.

Ports may also provide for ethernet and landline connectivity to exchange information with local and remote sources. For example, in the embodiment shown in FIG. 4, the system 300 is operable to communicate with a local or remote server 392. In a preferred embodiment, the server is a remote or cloud-managed server which is accessed by the computer system 310 as described herein. Software, data, and other information, as described herein, may be conveniently sent or uploaded to the workstation 310 from outside the operating room, and if desired, from a remote offsite location. In a preferred embodiment, the server 392 sends pre-operative 3D image data of the patient organ to the computing system. Examples of pre-operative data include, without limitation, CT, MRI, and other 3D image data of the patient, target ID, and route information, each of which is obtained prior to the procedure. In embodiments, CT image data is obtained of the lungs while the patient is asked to provide a full inspiration or expiation and the pre-acquired 3D image data is annotated with the beathing point or phase.

The system 300 is also shown including display 390. In embodiments, output is provided to the display. As described herein, the display 390 is operable with the computing device 310 to present reports, data, images, results, models, and views in various formats including, without limitation, graphical, tabular, and pictorial form. Additionally, in embodiments where the display is a touchscreen or tablet device, the screen can function as a user interface as well as a display.

### Software

The workstation 310 is shown including various software modules 340.

As described herein, and e.g., with reference to step 220 of FIG. 5, an image set select module 344 is operable to group 2D image slices from the ultrasound probe across multiple breath cycles into sets based on breath phase or point within a breath cycle. For example, 2D image slices of the same breath phase or point are grouped together. The number of groups or sets per breathing cycle can vary. In embodiments, the image slices are grouped into 2, 3, 4, 5, 6, 7, 8, 9, or 10 sets. In embodiments, the sets are grouped into 5-10, or upwards of 10 sets.

The software 340 is also shown including a 3D volume reconstruction module 348. The 3D volume reconstruction module 348 is operable to reconstruct 3D volume of the lung and other anatomy based on the ultrasound image data from the ultrasound probe 372, as described herein. In preferred embodiments, the 3D reconstruction image volume comprises motion or a sequence corresponding to the patient's breathing cycle. In this 4D imaging embodiment, the lung and anatomy can be computed as a volume over time showing the motion of the lung from complete inspiration to complete expiration and vice versa.

The software 340 is also shown including a registration module 350. The registration module 350 is operable to match the reconstructed 3D volume with pre-operative image data of the patient as described with reference to step 840 in FIG. 8. The registration generates a transformation map to deform and match the pre-acquired image data to the live 3D ultrasound reconstructed image data.

Optionally, the workstation can include a route planning module that is operable to compute a route to the target or region of interest based on the pre-operative data and user input as described herein.

Detector. The software 340 is also shown including a device tip computation module 352. The device tip computation module 352 is operable to detect and track the location of the device tip based on the ultrasound imaging and the 3D reconstructed image volume as described with reference to step 930 in FIG. 10.

Training. In embodiments, a detector is trained to recognize the surgical device in the reconstructed ultrasound image data. Multiple 3D images are prepared as the training data set showing surgical device in different positions. The hyperparameters of the detection algorithm are adjusted to optimize detection of the surgical device in the images.

The software 340 is also shown including a rendering module 354. Examples of items the rendering module 354 is operable to compute include without limitation, the surface or skeletal outline of one or more of the anatomies of the 3D reconstructed 3D volume, the planned route, the pre-operative image data, the device or device tip, the region of interest, as well as user annotations. The user may adjust the view, location, point in the breath cycle, that is being rendered and displayed and the rendering module is operable to recompute or update the image(s) to display based on the stored 3D reconstructed image volume, pre-operative image data, planned route, and device tip location.

Still other software may be included in the computer workstation 310 except where excluded in any appended claims.

### ALTERNATE EMBODIMENTS

The invention is intended to include various alternative embodiments.

For example, in embodiments, the imaging and guidance systems described herein are directed to non-lung organs that are visually observable via ultrasound, and preferably using noninvasive ultrasound. Examples of other non-lung organs include, without limitation, liver, kidney, bladder, pancreas, appendix, breast, heart, stomach, colon, trachea, esophagus, prostate, uterus, and generally speaking, the thorax and gastrointestinal regions of the body.

In another embodiment of the invention, robotic arms or automated mechanical assemblies control the ultrasound probe motion. For example, in an embodiment, a fixture adapted to hold the transducer is controlled by a linear motor and rail to move the ultrasound transducer across (or otherwise, e.g. tilt) the patient's chest. The linear motor & rail apparatus is controlled by the workstation to move according to a predetermined motion profile, e.g., a constant speed. The position information of the motor is used for localization and probe tracking as described above in addition to (or in lieu of) an optical tracker. The robotic motion can have some advantage where precise and repeatable motion is desired versus manual motion. Optionally, the mechanical and robotic assemblies are adapted to move the ultrasonic probe in several directions and/or multiple degrees of freedom including XYZ, as well as rotation, tilt, etc.

In another embodiment of the invention, and in the event various internal lung structures (e.g., blood vessels, airways, etc.) are not visible in the ultrasound images, a system and method are programmed and operable to estimate the location of the so-called invisible lung structures based on a pre-operative 4D imaging (e.g., 4D lung CT). In embodiments, the intra-breathing deformation of those lung structures are based on the pre-operative 4D imaging (e.g., 4D lung CT) and the location of the lung structures are estimated in the intra-operative 4D ultrasound image frames after the image registration. In embodiments, the pre-operative 4D image data is registered to the 4D ultrasound image data by framewise matching at one or more points during the patient breathing cycle (e.g., full inspiration, exhale, and midpoint).

In another embodiment of the invention, and in the event various internal lung structures are not visible and 4D image data of the patient is not available, a deformation model is created for the inner lung structures (such as airways and blood vessels) using 4D imaging datasets of several patients (e.g., a global or atlas-type model arising from numerous patients). The position of the invisible lung structures can be identified for each preoperative patient data set, all ranges can be aggregated, and used to compute typical locations (or ranges) for the invisible lung structures in an intra-operative procedure. In some embodiments, more sophisticated statistical models can be generated including machine learning or deep learning based modeling where the model is trained based on the preoperative patient 4D data sets. In these models, the local deformation of lung tissue during breathing can be represented by a vector space indicating the magnitude of displacement at each point over time or breathing phase. Then, for a new patient, the model is applied by selecting the closest image and breathing phase in the statistical model to the patient's image to estimate the deformation of the lung structures in the patient during breathing. Examples of references describing 4D deformable image registration of lung include: Castillo E, Castillo R, Martinez J, Shenoy M, Guerrero T. 2009. Four-dimensional deformable image registration using trajectory modeling. Phys Med Biol 55 305-327; and Castillo R, Castillo E, Guerra R, Johnson V E, McPhail T, Garg A K, Guerrero T. 2009. A framework for evaluation of deformable image registration spatial accuracy using large landmark point sets. Phys Med Biol 54 1849-1870.

Still in another embodiment, a non-transitory computer readable medium includes a set of instructions for use with a computer and is operable to carry out any one or combination of the steps, functions, and methods described herein.

Still in another embodiment, the 3D image data is not “pre-operative” and can be obtained during the same operation as the diagnostic or treatment is performed. For example, one can acquire a 3D image data set of the lung using cone beam CT (or another means) prior to the step of generating the ultrasound 2D images while still being considered part of the same operation. This pre-acquired 3D image data set can then be registered with the ultrasound 2D images, described herein.

While preferred embodiments of this disclosure have been shown and described, modifications thereof can be made by one skilled in the art without departing from the scope or teaching herein. The embodiments described herein are exemplary only and are not intended to be limiting. Because many varying and different embodiments may be made within the scope of the present inventive concept, including equivalent structures, materials, or methods hereafter thought of, and because many modifications may be made in the embodiments herein detailed in accordance with the descriptive requirements of the law, it is to be understood that the details herein are to be interpreted as illustrative and not in a limiting sense.

1. A system for registering real time 2D ultrasound data of a lung of a patient to pre-acquired 3D image data of the lung of the patient, the system comprising:
a storage on which the pre-acquired 3D image data of the lung of the patient is saved;
a processor programmed and operable to:
(a) reconstruct 3D image volumes from ultrasonically generated 2D image slices and position-time information for each of the 2D image slices; and
(b) register the pre-acquired 3D image data to the 3D image volumes.

2. The system of claim 1, further comprising an ultrasound scanner for generating the 2D image slices of the lung.

3. The system of claim 2, wherein the ultrasound scanner comprises a linear or phased-array ultrasound transducer.

4. The system of claim 2, further comprising a tracker system, the tracker system comprising at least one tracking marker on the ultrasound transducer, at least one tracking marker on the body of the patient, and a tracking sensor for detecting the position each of said tracking markers as a function of time.

5. The system of claim 4, wherein the tracking system is optical-based.

6. The system of claim 1, wherein the pre-acquired 3D image data is CT, MRI, or cone-beam.

7. The system of claim 1, wherein registration is performed by a patch matching registration algorithm.

8. The system of claim 1, wherein the 2D image slices are generated over a plurality of breathing cycles, and preferably at least four breathing cycles.

9. The system of claim 8, wherein the processor is operable to categorize the 2D image slices into different sets.

10. The system of claim 9, wherein the processor is operable to select a first set of 2D image slices for reconstructing a first 3D image volume based on a point in the breathing cycle.

11. The system of claim 1, further comprising a display, and the processor operable to show on the display the 3D reconstructed image volumes.

12. The system of claim 11, wherein the processor is further programmed and operable to detect and track a surgical device being advanced in the lung, and to display the surgical device in the 3D reconstructed volume.

13. The system of claim 12, wherein the processor is further programmed and operable to display a route to a target or region of interest in the 3D reconstructed volume.

14. The system of claim 13, wherein the processor is further programmed and operable to compute said route in the 3D reconstructed volume.

15. The system of claim 12, wherein the surgical device is a transthoracic or transbronchial aspiration needle, or ablation probe or catheter.

16. The system of claim 1, wherein the processor is further programmed and operable to calibrate the ultrasound probe location with the pre-operative 3D image data coordinate system.

17. The system of claim 1, wherein the processor is further programmed and operable to compute a suggested location, and optionally a suggested angle, for the ultrasound probe for generating the 2D image slices.

18. The system of claim 17, wherein the computing the suggested location and angle is based on location of airways in the lung.

19. The system of claim 18, wherein the computing the suggested location and angle is further based on lung motion.

20. The system of claim 19, wherein the system is operable to display the suggested location and angle, and optionally alert the physician if the probe is off-track or off-angle.

21.-48. (canceled)
